(19) United States
(12) Patent Application Publication
(10) Pub. No.: US 2022/0000953 A1
(43) Pub. Date: Jan. 6, 2022

(54) Antihypertensive Composition

(71) Applicants: Marcus KRUSE, Essen (DE); Tienush RASSAF, Essen (DE)

(72) Inventors: Marcus KRUSE, Essen (DE); Tienush RASSAF, Essen (DE)

(21) Appl. No.: 17/291,270

(22) PCT Filed: Jul. 29, 2019
(86) PCT No.: PCT/EP2019/070382

§ 371 Date: May 4, 2021

(51) Int. Cl. A61K 36/21; A61K 31/522; A61K 31/26; A23L 33/15; A23L 33/105; A61P 9/12

(52) U.S. Cl. CPC A61K 36/21; A61K 31/522; A61P 9/12; A23L 33/15; A23L 33/105; A61K 31/26

(57) ABSTRACT

The present invention relates to a composition having a concentration-promoting and/or stimulating effect, in particular for the simultaneous lowering of arterial blood pressure, wherein said composition contains as component (a) at least one nitrate source and/or nitrate and as component (b) at least one substance having a stimulating and/or stimulating effect, preferably caffeine.

### CROSS-REFERENCE TO RELATED APPLICATIONS

This application is a national stage application under 35 U.S.C. 371 of PCT Application No. PCT/EP2019/070382 having an international filing date of 29 Jul. 2019, which designated the United States, which PCT application claimed the benefit of European Application No. 18204420.6, filed 5 Nov. 2018, each of which are incorporated herein by reference in their entirety.

### SUMMARY

The present invention relates to the medical field of cardiovascular health.

In particular, the present invention relates to a composition which has a stimulating and concentration-promoting effect and at the same time leads to a reduction in arterial blood pressure. Furthermore, the present invention also relates to the uses of the compositions according to the invention.

In industrialized countries, cardiovascular diseases or diseases of the cardiovascular system are the most common cause of death. In recent years, cardiovascular diseases make up approximately 40% of the total causes of death in Germany, based on the respective year. The major risk factors for the development of cardiovascular diseases are excessive tobacco and alcohol consumption, obesity, and an unhealthy or unbalanced diet. Lack of exercise, elevated LDL cholesterol, diabetes mellitus and stress are also closely related to cardiovascular disease. The aforementioned risk factors favor the development of arterial hypertension (high blood pressure). High blood pressure stresses the cardiovascular system and significantly increases the risk of cardiovascular diseases.

Normal blood pressure values in adults are approximately 120/80 mmHg (“120 to 80”), wherein lower values are generally not harmful, provided they don't come along with symptoms that restrict quality of life, such as dizziness, increased pulse, fainting, fatigue, cold hands and feet, and concentration problems. Low blood pressure (hypotension) is defined as a blood pressure value of less than 100/60 mmHg. Very low blood pressure may be associated with dizziness, listlessness, fainting, fatigue, and cold extremities. High blood pressure or arterial hypertension is said to occur at a blood pressure value of 140/90 mmHg.

With regard to the values, it should also be noted that blood pressure generally increases somewhat with age, as the elasticity of the vessels decreases. Blood pressure values slightly above normal in older people or seniors can therefore be tolerated, provided there are no other risk factors for cardio-vascular diseases. Nevertheless, blood pressure should not be too high in the elderly people in order to avoid unnecessary stress on the cardiovascular system.

All of the aforementioned risk factors are particularly linked with the development of atherosclerosis (arteriosclerosis), i.e. a pathological accumulation of cholesterol esters and other fats in the inner wall layer of arterial blood vessels, which in turn is a major risk factor for heart attacks, strokes, occlusive diseases and aneurysms.

In order to lower elevated blood pressure and thus also reduce the risk of atherosclerosis, the initial focus is on non-drug measures and treatment concepts. Attempts are made to lower blood pressure by means of a healthy lifestyle based on a balanced diet which is rich in vitamins and fiber, especially in order to induce weight loss, as well as regular exercise. As a rule, however, such measures are only sufficient in the case of slightly elevated blood pressure, whereas in more severe cases, i.e. moderate to severe hypertension, the necessary reduction in blood pressure can no longer be achieved on this basis.

If the aforementioned lifestyle or habit changes are not sufficient to significantly lower blood pressure, drug therapy is inevitable.

Various blood pressure-lowering agents, collectively known as antihypertensives, are generally used for the drug therapy of high blood pressure:

ACE inhibitors or AT-1 receptor antagonists are used for their vasodilatory effect, which is based on the inhibition of the blood pressure-increasing hormone angiotensin II.

In addition, diuretics or diuretic agents are used to promote the excretion of salt and water via the kidneys. In combination with other antihypertensive drugs, their effect is enhanced.

In addition, so-called beta blockers are often used, which block the beta receptors, thereby inhibiting the effect of stress hormones (norepinephrine, epinephrine). By blocking the effect of stress hormones, the pulse and blood pressure decrease. As a result, the heart beats more slowly overall and is relieved.

Similarly, alpha-blockers or alpha-receptor blockers are also used, which have a vasodilatory and thus blood pressure-lowering effect. The effect is based on a competitive inhibition of the alpha1-adrenoreceptors.

Finally, calcium antagonists or calcium channel blockers may be used, which block the calcium channels in the heart and vascular muscle cells. As a result vascular tension is reduced and thus blood pressure is lowered.

The aforementioned therapeutics are used in the treatment of hypertension both as monotherapy and as combination therapy.

However, the problem with the aforementioned antihypertensive drugs is that they have a high potential for side effects. For example, ACE inhibitors are often accompanied by an unpleasant cough. In addition, angioedema, i.e. a swelling of the skin or mucous membranes, may develop. In rare cases, renal dysfunction may also occur or may be exacerbated. Calcium antagonists, on the one hand, can lead to so-called “flushing”. This causes the skin to redden and feel warmer, especially on the face. In addition, headaches, palpitations and water retention may occur. The frequently administered beta blockers, on the other hand, can lead to constriction of the bronchial tubes and also slow down the heart rate (bradycardia). In addition, circulatory disturbances in the extremities may also occur. Fatigue, depressive moods, dizziness and sleep disturbances are also possible. The administration of diuretics or dehydrating agents can also lead to a reduction of the sodium and/or potassium level in the blood, which in the worst case can end in life-threatening hyponatremia or hypokalemia.

Another disadvantage of drug therapy for high blood pressure is that it can take a long time to adjust to a suitable or optimal amount or dosage of the therapeutics. In the case of overdoses in particular, there is an excessive drop of blood pressure, which can be associated with dizziness, concentration problems and listlessness.

In light of the above, it is evident that there is a need in the prior art for therapeutics or agents that have few side effects and are well tolerated, which lead to a reduction in blood pressure and are suitable for the prevention or treatment of cardiovascular diseases.

Therefore, it is an object of the present invention to provide compositions or therapy concepts which overcome or at least reduce the aforementioned disadvantages of the concepts known from the prior art.

In particular, the present invention is based on the object of providing compositions or therapeutic concepts for the treatment of hypertension or for the prevention of cardiovascular diseases, which have fewer side effects, preferably essentially no side effects, compared to the prior art, and which are improved overall in their properties and effects.

To solve the object described above, the present invention proposes—according to the first aspect of the present invention—a composition according to claim 1; further, in particular advantageous embodiments of the composition according to the invention are the subject-matter of the respective subclaims.

Furthermore, the present invention relates—according to a second aspect of the present invention—to the uses of the compositions according to the present invention.

It is understood in connection with the remarks that follow that configurations, embodiments, advantages and the like, which are described below for the purpose of avoiding repetition only in relation to one aspect of the invention, of course also apply accordingly with respect to the other aspects of the invention, without this requiring separate mention.

With respect to the relative or percentage weight-related data mentioned below, in particular quantity data, it should furthermore be noted that these are to be selected by the skilled person in the context of the present invention in such a way that they always add up or add up to 100% or 100% by weight in total, taking into account all components or ingredients, in particular as defined below; however, this is also understood by the skilled person.

Furthermore, a person skilled in the art—depending on the application or particular case—can deviate from the weight, quantity and range data given below without leaving the scope of the present invention.

In addition, it applies that all values or parameters or the like mentioned in the following can basically be determined with standardized or explicitly stated determination methods or otherwise with determination or measurement methods familiar to the skilled practitioner in this field.

### BRIEF DESCRIPTION OF THE DRAWINGS

FIGS. 1A-1C: show the systolic blood pressure values obtained during the blood pressure measurements before and two hours after consumption of the juices, averaged over all subjects in the group.

FIG. 2: shows the effect of beet juice as a function of baseline blood pressure based on a comparison of the maximum change in blood pressure in the comparison group with low blood pressure and the other subjects with normal to high blood pressure.

FIGS. 3A-3C: show the results of the blood test with regard to the average nitrate and nitrite concentration in the plasma of the test subjects.

FIGS. 4A-4B: show the specific effects of composition A (group 1, white beet) on systolic blood pressure.

### DETAILED DESCRIPTION

Having said this, the present invention will now be explained in detail below.

Thus, the present invention provides—according to a first aspect of the invention—a composition with a concentration-promoting and/or stimulating effect, in particular for simultaneously lowering arterial blood pressure, wherein said composition comprises

(a) as component (a) at least one nitrate source; and

(b) as component (b) at least one substance having a stimulating and/or energizing effect, preferably caffeine.

In the context of the present invention, a completely surprising composition was found, wherein said composition increases on the basis of the combination of nitrate and a component with a stimulating or excitatory effect, in particular caffeine, the ability to concentrate or has a stimulating effect, but at the same time lowers arterial blood pressure.

In other words, the composition according to the invention allows to combine two fundamentally different or actually mutually exclusive effects in a single composition. Usually, stimulating active ingredients, such as caffeine, lead to an increase in pulse and blood pressure, while antihypertensive agents do not promote concentration or stimulation, but rather have a calming effect and slow down the heart rate.

In the context of the present invention, the reduction in blood pressure—without wishing to be bound to this theory—is achieved by the nitrate contained in the composition according to the invention. On the basis of a preferably oral intake of nitrate or topical application of nitrate in the oral cavity, blood pressure can be significantly lowered. The ingestion of nitrate leads to improved vascular function, i.e. the blood vessels, in particular the arteries, are dilated by the ingestion of nitrate, which in turn is associated with more efficient blood flow and leads to a relief of the cardiovascular system.

The effect of nitrate as an antihypertensive agent is based—again without wanting to be bound to this theory—on the NO metabolism, which leads to the reduction of nitrate (NO₃⁻) via nitrite (NO₂⁻) to nitrogen monoxide (NO). When nitrate is ingested orally, it is already reduced to nitrite in the mouth by bacteria or microorganisms of the oral flora and mucosa. After swallowing the saliva, the nitrite enters the bloodstream, where it is in turn enzymatically converted by nitrite reductases into nitric oxide. Nitric oxide is a signaling molecule that plays a central role in the human body with respect to vascular homeostasis. In particular, nitric oxide acts as a vasodilator and prevents rapid growth of vascular smooth muscle cells. An increase in blood NO levels significantly reduces vasoconstriction. In addition, nitric oxide also participates in neurotransmission, immune defense, and inhibition of platelet aggregation. Thus, by promoting the level of NO in the blood, an overall natural reduction in blood pressure can be achieved.

The present invention has numerous other advantages and special features, which are listed below and are to be regarded as indication of the existence of an inventive step:

As previously stated, it was completely surprising that, on the basis of the composition according to the invention, two contrary effects, namely blood pressure-lowering on the one hand and stimulating or concentration-promoting on the other, can be combined in a common composition. In particular, it was surprising in this context that the addition of a stimulating, energizing or concentration-promoting substance, preferably caffeine, does not lead to a compensation of the blood pressure-lowering effect of the nitrate or nitrate source. For, stimulating substances, such as caffeine, usually lead to an opposite effect of antihypertensive substances, namely an increase in heart rate and blood pressure.

Considering that the composition according to the invention is based on natural, well-tolerated ingredients, the intake of the composition is at least essentially free of undesired side effects. In particular, the side effects accompanying drug therapy of arterial hypertension, e.g. cough due to ACE inhibitors, listlessness, a slowed heart rate, dizziness, fatigue, etc., do not occur when consuming the composition according to the invention. Thus, on the basis of the composition according to the invention, an effective and non-drug treatment or prevention of hypertension is possible, while typical side effects occurring in drug therapy are avoided. Overall, the composition according to the invention has a higher overall tolerability.

In addition, there is no risk of incorrect or excessive dosages with the composition according to the invention. This is because—as the examples of embodiments described below will show—the effect of lowering blood pressure only occurs in patients who actually have a blood pressure that is too high or a blood pressure in the range of the upper normal values. In other words, the blood pressure lowering effect only occurs at an elevated basal blood pressure. Furthermore, an increase in the dose/amount of active ingredients does not lead to a further reduction in blood pressure. However, the blood pressure is never lowered too much, i.e., there is no lowering into the range of a low blood pressure of less than 100/60 mmHg. Thus, when the composition according to the invention is taken at normal or low blood pressure, it does not promote any further blood pressure-lowering effect. Persons with “healthy blood pressure” can thus also safely consume the composition according to the invention and benefit from the concentration-promoting or stimulating effect without further lowering of the blood pressure.

Furthermore, the composition according to the invention can be present in a wide variety of dosage forms, whereby it is preferred when the compositions according to the invention are compositions intended for consumption or compositions which are prepared for topical application in the oral cavity. This is because, as previously stated, nitrate is already reduced in the oral cavity by nitrate-reducing microorganisms and bacteria of the oral flora to nitrite, which in turn is swallowed with the saliva and thus enters the bloodstream for the generation of nitric oxide.

Particularly good results are obtained with respect to the effects of the compositions according to the invention when the compositions are in the form of beverages (cf. working examples). In addition, however, it is equally possible to provide the compositions in particular in the form of chewing gums, mouth rinses or mouthwashes, or toothpaste.

The composition according to the invention is thus also particularly suitable for use as a food (supplement) to support cardiovascular health without side effects, and to lower blood pressure or to prevent high blood pressure.

With regard to the effects of the compositions according to the invention, reference is already made at this point to the working examples described in detail below, which prove the effects of the compositions according to the invention, in particular with regard to the lowering of blood pressure, and which are to be regarded as an indicator of the existence of an inventive step.

In the following, definitions of the terms used in the context of the present invention are given for a better understanding of the composition or concept according to the invention and, in addition, particularly preferred embodiments of the present invention are described.

The term “composition” as used in the context of the present invention is to be understood very comprehensively and refers, on the one hand, to pharmaceutical or medical preparations and, on the other hand, also to food compositions, such as beverages or other food (supplement) products intended for oral consumption, but furthermore also to cosmetics or hygiene articles or drugstore products, such as dental/oral care products, for example chewing gums or mouth rinses.

In the context of the present invention, the term “nitrate source” refers to substances which are suitable for consumption or pharmaceutically acceptable and contain nitrate, in particular inorganic nitrate salt or organic nitrates, such as plants, vegetables, etc. with a high nitrate content. Similarly, a nitrate source within the meaning of the present invention may also be formed by nitrates as such, in particular inorganic nitrate salt or organic nitrates. In the context of the present invention, it is particularly preferred if the nitrate source is of plant origin, i.e. when components of plants which accumulate or store nitrate are used as the nitrate source. Plants which contain large amounts of nitrate include, among others, beets (beet), chard, spinach, various lettuces, cress and cabbage varieties. As will be explained below, vegetable nitrate sources can be used in the form of liquid components of the plants, in particular juices or extracts obtained from plants or plant components. In addition, it is also possible to use solid plant components as a nitrate source, for example ground and powdered components, such as roots, leaves or storage organs.

The term “nitrate” is understood in the context of the present invention to mean the salts and esters of nitric acid (HNO₃) of nitrate salts having the general composition M^{I}NO₃ (M^{I}: monovalent cation). Usually, the monovalent cation is an alkali metal salt, in particular potassium or sodium. Similarly, nitrate salts based on nitrate anions and alkaline earth metals, in particular magnesium, calcium or barium, can also be formed. Esters of nitric acid are organic nitrate compounds which have the general structure R—O—NO₂ (R: organic radical). For further details on the term “nitrate”, reference is made to RÖMPP Chemie Lexikon, 10th edition (1998), Georg Thieme Verlag, keyword: “nitrates”, page 2911, the disclosure described therein being hereby incorporated by reference.

The term “stimulating or energizing substance” refers in particular to psychotropic substances that increase, accelerate or improve the activity of the nerves so that concentration, alertness and energy levels are increased.

According to the invention, it is particularly preferred when caffeine is used as the stimulating or energizing substance. Caffeine (synonymously also known as theine) is an alkaloid from the xanthine group. The chemical name of caffeine is 1,3,7-trimethylxanthine. Caffeine is a psychoactive substance with a stimulating effect and is primarily obtained from the beans of coffee plants (Coffea) or leaves of the tea bush (Camellia sinensis). The spectrum of action of caffeine is very broad. The main effects of caffeine are stimulation of the central nervous system, increase in the contractility of the heart, pulse increase, bronchial dilation, slight increase in blood pressure and stimulation of peristalsis of the intestine. However, when relatively small doses of caffeine are used, the stimulating effect based on stimulation of the central nervous system is predominant. By stimulating the central nervous system, the use of caffeine can increase attention and concentration.

As previously stated, the combination of active ingredients according to the invention provides a composition which, in a completely surprising way, combines two opposing effects or effects in a single composition, namely a concentration-promoting and stimulating effect on the one hand and a reduction in arterial blood pressure on the other hand.

The composition according to the invention can be designed in a variety of ways, with preferred embodiments being described below.

As for the amount of nitrate used in the compositions according to the invention, this can vary within wide ranges. In order to achieve a good efficacy in lowering blood pressure, it has been shown to be advantageous when component (a) and/or the nitrate source contains nitrate in an amount, based on the weight-related amount of NO₃ and/or NO₃⁻per serving and/or application unit of the composition, in the range of 1 to 10,000 mg, in particular 10 to 5,000 mg, preferably 100 to 2,500 mg, preferably 200 to 1,000 mg, particularly preferably 300 to 700 mg.

With nitrate supplementation or application of nitrate in the oral cavity in the above-mentioned quantity ranges, a reduction in systolic blood pressure of up to 15 mmHg can be achieved. As previously stated, the effect of nitrate depends on the basal blood pressure, i.e., the higher the basal blood pressure, the better the blood pressure-lowering effect of the compositions according to the invention. If, on the other hand, the blood pressure is low to normal, the compositions according to the invention will not lower the blood pressure any further.

In the context of the present invention, it may be provided that component (a) and/or the at least one nitrate source is selected from vegetable nitrate sources and/or salts and/or esters, in particular salts, of nitric acid and/or mixtures thereof, preferably vegetable nitrate sources.

According to a particularly preferred embodiment, it is thus preferred that component (a) is based on a nitrate source of plant origin, i.e. that the composition contains as component (a) at least one vegetable or plant-based nitrate source. According to this particular embodiment of the present invention, the nitrate source may be selected in particular from plants belonging to the group of Spinacia oleracea (vegetable spinach), Raphanus (radish), Beta vulgaris (common turnip), Brassica rapa (turnip), Diplotaxis tenmfolia (arugula), Lepidium (cress), Valerianella (lamb's lettuce), preferably Beta vulgaris (common turnip). The aforementioned plants, especially their leaves and storage organs, are rich in nitrate and may have a nitrate content of more than 1,000 mg/kg.

Particularly good results are achieved when the vegetable nitrate source is based on components of Beta vulgaris, which includes diverse subspecies and cultivated forms. Beta vulgaris or common beet is a plant species of the amaranth family with numerous subspecies, which include sugar beet, various cultivated forms of beet, chard and fodder beet. The common beet is descended from the wild beet, which has an extensive range from Western Europe to Western Asia. With regard to the present invention, the use of Beta vulgaris based on the cultivated forms “beet”, “white beet” or “yellow beet” is particularly preferred. The content of nitrate, especially in the storage organs, is usually in the range of 1,000 to 4,000 mg/kg. The aforementioned cultivated forms of beet also have a high vitamin B, potassium, iron and folic acid content.

According to a particularly preferred embodiment of the composition according to the invention, it may thus be provided that the composition comprises as component (a) at least one vegetable nitrate source, the vegetable nitrate source being obtained from the yellow, red and/or white cultivated form, preferably from the yellow and/or white cultivated form, of the common beet (Beta vulgaris). With regard to the antihypertensive effect, the aforementioned varieties of beet are equally suitable. However, the use of light beet in particular, i.e. yellow or white beet, has proved advantageous in that these are essentially free from the deep red natural dye betanin. The disadvantage of this dye is that it can enter the urine when consumed, leading to a red coloration of the urine. One problem with urine dyed red by betanin is that it makes laboratory diagnosis of a patient's urine more difficult, since betanin interferes with chemical-biological analysis. On the other hand, red-colored urine sometimes leads to confusion among patients or consumers of the compositions, as they are not always aware that the coloring is harmless and merely due to the dye contained in bete, and that it is not blood or other symptoms of disease. In this respect, it is particularly preferred in the context of the present invention if the vegetable nitrate source is based on yellow and/or white beet.

With regard to the embodiment, according to which component (a) is based on at least one vegetable nitrate source, it is particularly preferred when the composition contains the at least one vegetable nitrate source in the form of liquid plant constituents, in particular plant extract and/or plant juice, and/or in the form of solid plant constituents, in particular leaves, roots and/or storage organs in preferably ground form. Particularly good results are obtained, especially with regard to the provision of compositions in liquid form, such as beverages, if the plant nitrate source is used in the form of plant extract and/or plant juice. According to a particularly preferred embodiment of the present invention, the liquid nitrate source, e.g. beet juice, forms the basis for the composition (cf. also embodiments according to the invention), to which the other ingredients are then admixed.

According to a further embodiment of the present invention, it may also be provided that the composition contains as component (a) nitrate in the form of salts and/or esters of nitric acid, in particular salts of nitric acid. In this context, it is particularly preferred when component (a) is selected from the group of alkali metal and/or alkaline earth metal salts of nitric acid, in particular potassium nitrate, sodium nitrate, barium nitrate, calcium nitrate, magnesium nitrate and/or mixtures thereof, preferably potassium nitrate and/or sodium nitrate.

According to a further embodiment, it may further be provided that the composition according to the invention comprises as component (a) a combination of a nitrate source of vegetable origin, such as beet juice, and inorganic nitrate, e.g. based on potassium or sodium nitrate.

As for component (b) based on a substance having a stimulant or stimulating effect, it is particularly preferred in the context of the present invention when the composition contains caffeine as component (b).

The amount of caffeine used is variable. Particularly good results are obtained if the composition contains component (b), based on a portion and/or application unit of the composition, in an amount in the range from 1 to 1,500 mg, in particular 5 to 1,000 mg, preferably 10 to 500 mg, preferably 50 to 250 mg, particularly preferably 80 to 150 mg. On the basis of the above-mentioned quantities, a central nervous system-stimulating effect is achieved, in particular the ability to concentrate is increased, but without compensating or blocking the effect of the nitrate as a blood pressure-lowering agent.

In order to further improve the efficacy or the spectrum of efficacy of the composition according to the invention, it may further be provided that the composition according to the invention contains at least one mineral substance as component (c).

The term “minerals” is used in the context of the present invention as a collective term for the mineral constituents of plant and animal organisms, i.e., in particular, of humans. In the human body, the amount of minerals is about 3.5 kg. A distinction is made between so-called microelements with trace element character, which mainly have a catalytic function, and macroelements, which predominate in terms of quantity and which are indispensable as “building materials”, for example for the skeleton. The microelements include in particular fluorine, bromine, iodine, iron, copper, manganese, cobalt, zinc, molybdenum, vanadium and selenium. Macroelements, on the other hand, include calcium, sodium, potassium, phosphorus, sulfur, magnesium and chlorine. For further details on the term “minerals”, reference is also made to RÖMPP Chemie Lexikon, 10th edition (1998), Georg Thieme Verlag, Stuttgart New York, keyword: “Mineralstoffe”, pages 2700 to 2701. The disclosure content described therein is moreover hereby incorporated by reference into the disclosure content of the present invention.

According to a preferred embodiment of the present invention, it may thus be provided, with respect to the use of a mineral, that component (c) is selected from the group of calcium, chlorine, potassium, magnesium, sodium, phosphorus, sulfur, iron, iodine, copper, manganese, molybdenum, selenium, zinc and/or mixtures thereof, preferably magnesium.

Magnesium is an alkaline earth metal which—as explained above—is a macro element for humans. The body of an adult human contains approx. 20 g of magnesium. Magnesium is involved in numerous enzymatic reactions in the human body; either as an enzyme component or as a coenzyme. In addition, free magnesium ions play a role in the transmission of stimuli in or at the nerve cells, as they influence the potential at the cell membranes, and also act as so-called “second messengers” in the immune system. Magnesium deficiency can be associated with various symptoms, such as restlessness, nervousness, irritability, lack of concentration, fatigue, a general feeling of weakness, headaches, cardiac arrhythmias and muscle cramps. Magnesium deficiency can thus be prevented or treated by the use of magnesium in the compositions according to the invention.

As far as the amount of component (c) or of the at least one mineral used is concerned, this can also vary within wide ranges. According to a preferred embodiment of the present invention, it is preferred when the composition contains component (c), based on a portion and/or application unit of the composition, in an amount ranging from 1 to 1,000 mg, in particular from 5 to 500 mg, preferably from 10 to 250 mg, more preferably from 50 to 200 mg.

According to a further preferred embodiment of the present invention, it may further be provided that the composition comprises as component (d) at least one vitamin and/or provitamin.

According to the present invention, vitamins are understood to be organic compounds which do not serve the organism directly as energy carriers, but which the organism rather requires with regard to other functions. However, the human organism cannot synthesize all vitamins itself. Instead, vitamins or precursors of vitamins (so-called provitamins) must be ingested with food and thus belong to the essential substances. In the case of provitamins or precursors, these are only converted into the physiologically active form after their intake. Basically, vitamins are divided into fat-soluble (lipophilic) and water-soluble (hydrophilic) vitamins. As far as the function of vitamins is concerned, they are involved in numerous metabolic reactions. In particular, vitamins regulate the utilization of carbohydrates, proteins and minerals, so that vitamins are also involved, at least indirectly, in energy production. Furthermore, vitamins support the immune system. In addition, vitamins help to build cells, blood cells, bones and teeth. Overall, vitamins are a chemically extremely heterogeneous group whose members also differ significantly from one another in terms of their function and effect. A total of 13 vitamins are known, namely vitamin A, the B vitamins B1, B2, B3, B5, B6, B9 and B12 (hereinafter collectively referred to as vitamin B), vitamin C, vitamin D, and vitamins E and K.

In the context of the present invention, it may thus be provided in particular that component (d) is selected from the group of vitamin A, vitamin B, vitamin C, vitamin D, vitamin E, vitamin K and/or mixtures thereof, in particular vitamin C.

Vitamin C (also known synonymously as ascorbic acid) is an organic acid with the chemical name 5-1,2-dihydroxyethyl-3,4-dihydroxyfuran-2-one. Vitamin C physiologically acts as a free radical scavenger due to its antioxidant activity. Vitamin C thus acts as a reducing agent in the body and is able to inactivate reactive oxygen species (ROS) or free radicals, resulting in a reduced risk of oxidative stress. Excessive oxidative stress caused by free radicals accelerates aging processes on the one hand and promotes the development of various diseases on the other. A deficiency of vitamin C is also associated with the vitamin deficiency disease scurvy. The use of vitamin C in the compositions according to the invention further extends and improves the mode of action of the composition according to the invention, since vitamin C—as previously explained—acts as a free radical scavenger and also prevents the vitamin deficiency disease scurvy.

Furthermore, as far as the amount of vitamins or component (d) used in the compositions according to the invention is concerned, this can also vary within wide ranges. However, particularly good results are obtained when the composition contains component (d), based on a portion and/or application unit of the composition, in an amount in the range from 1 to 5,000 mg, in particular 5 to 2,500 mg, preferably 10 to 1,500 mg, more preferably 50 to 1,000 mg, particularly preferably 100 to 500 mg, still more preferably 200 to 400 mg.

In order to further improve the spectrum of action of the composition according to the invention or to achieve an even broader mode of action, it may also be envisaged that the composition according to the invention contains as component (e) at least one substance with antioxidant activity, preferably indirect antioxidant activity. As previously stated, antioxidants or antioxidants are chemical compounds that slow down or prevent oxidation of other substances. On this basis, antioxidants act as radical scavengers, which inactivate reactive oxygen species, such as free radicals, and on this basis prevent or reduce oxidative stress. However, antioxidants can act in different ways, namely by directly neutralizing reactive oxygen species or free radicals or indirectly by activating substances which directly neutralize the free radicals or reactive oxygen species. While antioxidants based on vitamins, as already explained above, have a direct effect on free radicals, indirect antioxidants lead to an activation of the body's own defense mechanisms with regard to the prevention of oxidative stress.

In this context, it has proven advantageous, when component (e) or the substance with antioxidant activity is sulforaphane. Sulforaphane, chemically 1-isothiocyanato-4-methanesulfinyl-butane, is an isothiocyanate formed during the enzymatic hydrolysis of the glucosinolate glucoraphanin. In addition, sulforaphane occurs as a secondary plant substance based on glucoraphanin, particularly in cruciferae (Brassicaceae). Sulforaphane acts physiologically as a powerful indirect antioxidant. In the human body, sulforaphane activates so-called phase II enzymes, which in turn trigger antioxidant defense mechanisms. Through the additional use of sulforaphane in the compositions according to the invention, an independent antioxidant effect can thus be achieved or, if the use of vitamins, in particular vitamin C, is also envisaged, the direct antioxidant effect of the vitamins can be synergistically supplemented or enhanced by an additional indirect antioxidant.

With regard to the amount of component (e) and/or sulforaphane, it has also been shown to be advantageous when the composition contains component (e) and/or sulforaphane, based on a portion and/or application unit of the composition, in an amount in the range from 0.01 to 500 mg, in particular 0.1 to 250 mg, preferably 1 to 100 mg, more preferably 5 to 50 mg, particularly preferably 10 to 30 mg.

As previously stated, the composition according to the invention can be present in a wide variety of dosage forms. According to the invention, it is particularly preferred if the composition is prepared for consumption or is in the form of a composition to be applied in the oral cavity, such as mouthwash, toothpaste or chewing gum. This is because—as previously explained—the conversion of nitrate to nitrite already begins in the oral cavity by nitrate-reducing microorganisms.

Depending on the dosage form, the compositions can thus be designed differently and accordingly contain different excipients, as detailed below:

With regard to a composition which is chewable but not intended to be swallowed, or a composition in the form of chewing gum, it is advantageous when the composition contains chewing gum base as an excipient. In principle, when providing a composition in the form of chewing gum, the person skilled in the art knows which excipients or base materials are to be used therefor. According to a preferred embodiment in this respect, it may be provided that the chewing gum base comprises natural polymers, in particular rubber, preferably in the form of latex, crepe or sheets, polyols and mixtures thereof. Similarly, it may be provided that the chewing gum base comprises synthetic polymers, in particular synthetic thermoplastics, preferably polyvinyl ethers, in particular poly(ethyl vinyl ether) and/or poly (isobutyl vinyl ether), polyvinyl acetates, vinyl acetates, polyisobutenes and/or mixtures thereof.

When the composition according to the invention is provided in the form of chewing gums or chewable compositions, the nitrate or nitrate source is incorporated into the chewing gum base or the chewing gum base is coated with nitrate or the nitrate source, which is then released into the oral cavity during chewing. As previously explained, the released nitrate is reduced to nitrite by the microorganisms or bacteria present in the oral flora and is swallowed with the saliva to enter the bloodstream on this basis, where the reduction of nitrite to nitric oxide finally takes place by nitrite reductases.

Similarly—according to a further preferred embodiment—it may also be envisaged that the composition according to the invention is in the form of solid compositions, such as lozenges, candies, hard caramels, lollies or the like. In this context, it is advantageous if the composition comprises as excipient at least one solid carrier and/or matrix material. The provision of lozenge-shaped compositions or candies likewise presents no technical hurdles to the skilled person, i.e. the skilled person knows how to select the suitable carriers or matrix materials.

However, it is particularly preferred in this context if, for solid dosage forms, in particular based on candies, hard caramels, lozenges and the like, the carrier and/or matrix material is selected from sugars and/or sugar substitutes, preferably from the group of sucrose, glucose, dextrose, fructose, mannitol, xylitol, sorbitol, isomalt, maltitol syrup, lactitol, leucrose, fructooligosaccharides, glucans, polyglucose and/or mixtures thereof.

In this case, too, the active substances of the composition according to the invention are incorporated into the carrier or matrix material and are released into the oral cavity when the composition is sucked or chewed, reduced to nitrite and swallowed with the saliva, so that a further reduction to the nitric oxide which actually lowers blood pressure takes place.

Finally, it may also be envisaged that the composition according to the invention is present in liquid dosage form. According to the present invention, the term liquid dosage form has to be understood broadly. On the one hand, this may include beverages, such as energy drinks or juice preparations. On the other hand, the term liquid dosage forms also includes cosmetics or hygiene or drugstore articles, such as mouthwashes, mouth rinses and the like.

In this context, it is preferred when the composition contains at least one liquid carrier material. According to this embodiment of the present invention, the active ingredients or ingredients of the composition according to the invention are dissolved or solubilized in the carrier material. In principle, all pharmaceutically or nutritionally acceptable carrier materials are suitable here.

As previously stated in connection with the nitrate source, it is possible that the carrier material for liquid compositions is already formed by a liquid nitrate source, such as beet juice, to which the further ingredients are then added. This has proven to be particularly advantageous in relation to drinkable compositions.

Furthermore, in connection with other liquid compositions, e.g. in the form of mouthwash, it may also be provided that the carrier material contains polar and/or apolar solvents, preferably water, ethanol, glycerol, hydrogenated castor oil, polyethylene glycol, polypropylene glycol and/or mixtures thereof.

Finally, in view of the present invention, it may also be advantageous if the composition contains at least one further ingredient, in particular wherein the at least one further ingredient is selected from the group of additives and/or adjuvants, preferably binding, wetting, stabilizing, coloring, buffering, smelling, flavoring, sweetening, aroma, processing aids and preservatives and agents and/or mixtures thereof.

The use of further ingredients may be provided, in particular with regard to the taste of the composition according to the invention, in order to produce specific flavors or tastes or to mask undesired tastes. Undesired tastes can particularly result from vegetable nitrate sources. In addition, further ingredients may be included to further improve consistency, shelf life, stability, etc. of the composition according to the invention.

As previously stated, it is preferred according to the invention if the composition is prepared for consumption and/or for topical application, in particular in the oral cavity.

On the basis of such compositions, a reduction of nitrate to nitrite is already ensured in the oral cavity, with the nitrite being swallowed down with the saliva and further reduced to nitric oxide in the bloodstream. In the case of compositions intended for consumption, swallowed nitrate is also further converted to nitrite in the body, in particular by nitrate-reducing bacteria in the gastrointestinal tract, and likewise enters the bloodstream for further reduction to nitric oxide.

As far as the consistency of the composition according to the invention is concerned, the composition is preferably prepared in solid, liquid, viscous (gel-like), semi-solid, aerosol or pasty form, in particular liquid or solid form.

The person skilled in the art knows in principle how compositions can be provided in the various forms, which additives or auxiliaries are to be used and which technical measures are to be taken.

With regard to specific dosage forms, it may further be provided according to a preferred embodiment of the composition according to the invention that the composition is prepared as a beverage, bar, pastry, hard caramel, chewing gum, toothpaste and/or mouthwash (mouth rinse), in particular as a beverage, hard caramel, chewing gum or mouthwash, preferably as a beverage.

As far as the specific uses of the compositions according to the invention are concerned, these are variable. On the one hand, a pharmaceutical and/or medical use of the composition for the preventive or therapeutic treatment of hypertension is possible. Similarly, the compositions according to the invention can also be used as foodstuffs or food supplements, such as beverages, “energy drinks”, chewing gums, etc., hygiene products, such as mouth rinses, and the like.

According to a particularly preferred embodiment, it may thus be envisaged that the composition according to the present invention is intended for use in the preventive and/or therapeutic treatment of arterial hypertension (hypertension). On this basis, an essentially side-effect-free lowering of blood pressure can be achieved on the basis of natural active ingredients, as previously detailed.

In addition, it may also be envisaged that the composition according to the invention is used to increase the energy level and/or concentration. In particular, so-called “energy products” can be provided on the basis of the compositions according to the invention, e.g. on the basis of drinks, sweets, bars or chewing gums. Particularly preferred in the context of the present invention is the provision of “energy drinks” with a stimulating or stimulating and simultaneously blood pressure-lowering effect, as also described below in the context of the embodiments.

Overall, the present invention thus provides a versatile composition which can be used both in the medical field as a blood pressure lowering agent and in the “lifestyle field” as a food or dietary supplement or hygiene or drugstore product, and which combines two opposing effects, namely a blood pressure lowering effect, on the one hand, with a concentration-promoting or stimulating effect, on the other hand.

Furthermore, the present invention relates—according to a second aspect of the present invention—to the use of a composition as described above as a food-stuff, food supplement and/or sanitary article with a concentration-promoting and/or stimulating effect, in particular for simultaneously lowering arterial blood pressure.

When using the composition according to the invention as a foodstuff, food supplement and/or hygiene article with a concentration-promoting and/or stimulating effect, it is preferred in the context of the present invention that the composition is applied via the oral cavity, i.e. by oral ingestion or by topical application, e.g. based on an oral rinse solution. In this process—as already explained in detail above—the nitrate contained in the compositions is first reduced by nitrate-reducing bacteria of the oral cavity or in the gastrointestinal tract to nitrite, which in turn enters the bloodstream and is further reduced there to the blood pressure-lowering nitric oxide.

For further details on the use according to the invention, reference can be made to the preceding explanations on the composition according to the invention, which also apply accordingly with regard to the use according to the invention.

Preferred embodiments of the present invention are described below on the basis of the embodiment examples, which, however, are in no way to be understood as limiting.

### WORKING EXAMPLES

1. Manufacturing Examples

In order to investigate the efficacy of compositions according to the invention, particularly with regard to the antihypertensive activity, various vegetable nitrate sources (juice of red, white, and yellow beet each containing 7 mmol of nitrate (corresponding to a weight-based nitrate amount of 434 mg per application unit of 150 to 250 ml)) were first investigated as such with regard to their antihypertensive activity.

In addition, compositions A, B and C according to the invention were provided, which contained a vegetable nitrate source in liquid form based on Beta vulgaris in combination with further ingredients, namely caffeine, vitamin C, magnesium and sulforaphane. The following table shows the formulations for the provided compositions A to C:

TABLE 1
Formulations according to the invention

| Ingredient | Composition A | Composition B | Composition C |
|---|---|---|---|
| Nitrate source | White beet juice (150 | Yellow beet juice (100 | (Red) Beet juice (250 |
| | ml) | ml) | ml) |
| Nitrate content | 434 mg (7 mmol) | 434 mg (7 mmol) | 434 mg (7 mmol) |
| Stimulant | Caffeine (100 mg) | Caffeine (100 mg) | Caffeine (100 mg) |
| Vitamin | Vitamin C (250 mg) | Vitamin C (250 mg) | Vitamin C (250 mg) |
| Mineral | Magnesium (150 mg) | Magnesium (150 mg) | Magnesium (150 mg) |
| Indirect | Sulforaphane (25 mg) | Sulforaphane (25 mg) | Sulforaphane (25 mg) |
| Antioxidant | | | |
| Ratio nitrate/ | 4.34:1 | 4.34:1 | 4.34:1 |
| caffeine | | | |

The above compositions according to Table 1 were prepared by methods known per se to the skilled person. For this purpose, the other ingredients, i.e. vitamin C, magnesium, sulforaphane and caffeine, were added to the liquid nitrate source based on beet juice in the amounts listed in Table 1.

The determination of the nitrate quantity or the nitrate content in the nitrate sources was also carried out with methods familiar to the specialist, namely by high-performance liquid chromatography (HPLC, instrument used: ENO-20, Eicom, San Diego, USA).

2. Blood Pressure Lowering Effect of White and Yellow Beet

Before analyzing the influence on blood pressure of the compositions A to C according to the invention as shown in Table 1 with nitrate, caffeine and the other ingredients, the influence of the nitrate sources used in the compositions according to the invention based on beet juices from red, yellow and white beet without additional active ingredients, in particular without caffeine, on blood pressure has been determined. In addition, it was also investigated in this context whether juices from yellow and white beet have any blood pressure-lowering effect at all. Up to now, only (red) beet has been described in the prior art in connection with an antihypertensive effect.

In order to compare the antihypertensive effects of white, yellow, and red beet juices, each juice was provided with 7 mmol or 434 mg of nitrate per serving or application unit (150 to 250 ml). The nitrate concentration was determined by HPLC, as described above.

Study Design

A subject collective based on 30 male subjects aged 35 to 55 years was used to investigate the effect. All subjects were slightly overweight with a BMI between 27 to 30, smokers, and had blood pressure in the upper normal or high range. The 30 subjects were divided into three subject groups of ten subjects each, with each sample group testing one of the juices (group 1: beet, group 2: white beet, group 3: yellow beet). For comparison purposes, a further five subjects were also included, who had a low systolic blood pressure of 110 mmHg or less. Subjects in this comparison group received beet juice.

In the study, the subjects received one application unit of the respective beet juice containing 7 mmol or 434 mg of nitrate for consumption. To determine the blood pressure-lowering effect, systolic blood pressure was measured immediately before consumption of the beet juices. A second measurement was taken two hours (120 minutes) after consumption.

Results

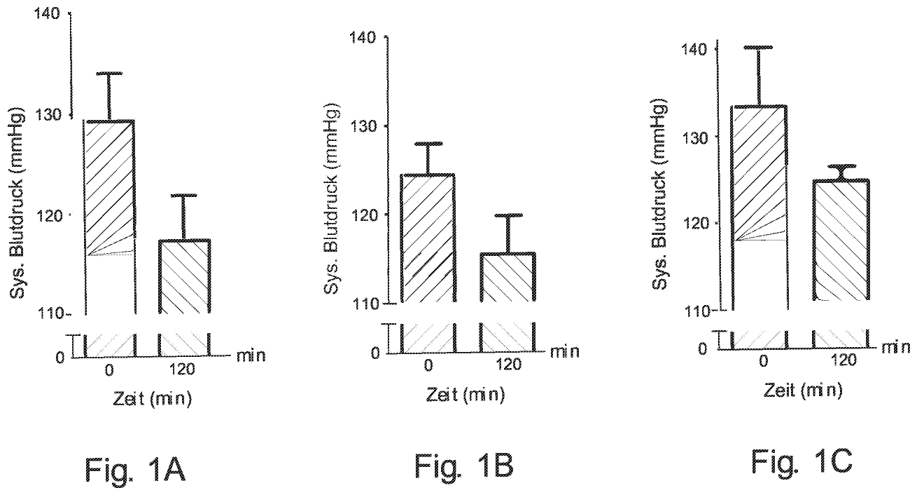

The systolic blood pressure values obtained during the blood pressure measurements before and two hours after consumption of the juices, averaged over all subjects in the group, are shown in FIG. 1 (FIG. 1A: (red) beet, FIG. 1B: white beet, FIG. 1C: yellow beet).

In group 1 (FIG. 1A), the systolic blood pressure of the test subjects was approximately 129 mmHg immediately before ingestion of the juice. Two hours after consumption, the blood pressure of the test subjects was on average significantly below 120 mmHg, i.e. a blood pressure reduction of more than 10 mmHg was achieved. In group 2 (FIG. 1B), which had received white beet juice, the average systolic blood pressure of the subjects before ingestion of the juice was approximately 124 mmHg, i.e. in the upper normal, non-optimal range. Two hours after consumption of the juice, the average systolic blood pressure of the subjects was only circa 115 mmHg and had thus fallen into the optimal range. Group 3 (FIG. 1C) received yellow beet juice. The average systolic blood pressure of the subjects in this group before consumption of the juice was approximately 133 mmHg. Two hours after consumption, blood pressure had fallen to average values of approximately 123 mmHg.

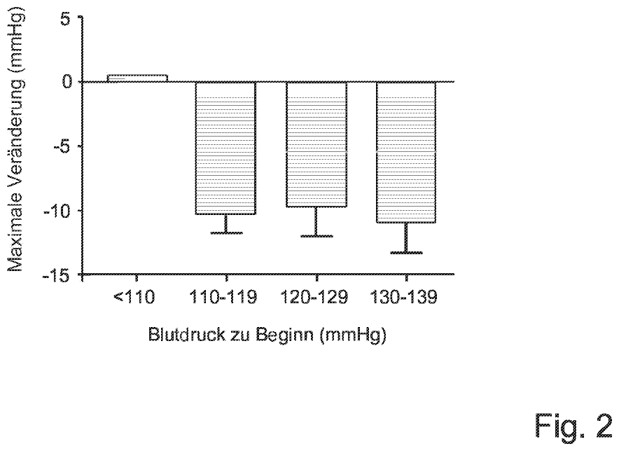

FIG. 2 also shows the effect of beet juice as a function of baseline blood pressure based on a comparison of the maximum change in blood pressure in the comparison group with low blood pressure and the other subjects with normal to high blood pressure. It can be seen that in subjects with low blood pressure of 110 mmHg maximum, no further reduction in blood pressure occurred. In the other subjects, however, blood pressure was reduced by an average of around 10 mmHg.

All the beet juices tested with a nitrate content of 434 mg per application unit or per serving led to a significant reduction in systolic blood pressure in the subjects. In this context, it has also been shown that the nitrate sources based on yellow and white beet preferred in accordance with the invention are equally suitable for achieving a reduction in blood pressure. In addition, it has been shown in the course of the study that the blood pressure-lowering effect only occurs when the blood pressure is in the normal or high range. Low blood pressure, on the other hand, is not lowered further.

3. Blood pressure lowering effect of the compositions according to the invention.

Furthermore, the effect of the compositions according to the invention listed in Table 1, which in addition to the nitrate source based on beet juice also contained caffeine as a stimulating substance and furthermore vitamin C, magnesium and sulforaphane, was also investigated by means of application studies. In this context, it should be analyzed in particular whether the addition of caffeine, which has a stimulating effect contrary to that of nitrate, cancels out the blood pressure-lowering effect of the nitrate source based on beet juice.

Study Design

A subject collective based on 12 male subjects aged 35 to 55 years was used to investigate the effect. All subjects were slightly overweight with a BMI between 27 to 30, smokers and suffered from arterial hypertension. The 12 subjects were divided into three subject groups of four subjects each, with each sample group testing one of the compositions A to C (group 1: composition A, group 2: composition B, group 3: composition C).

During the study, the subjects received one application unit of the respective composition containing 7 mmol or 434 mg of nitrate for consumption. To determine the blood pressure-lowering effect, systolic blood pressure was measured immediately before consumption of the respective composition. A second measurement was taken two hours (120 minutes) after consumption. In addition, the concentration of nitrate and nitrite in the blood plasma of the subjects was examined before consumption of the compositions according to the invention and two hours after administration of the composition. The measurement of nitrate and nitrite concentration in the plasma of the subjects was also performed by HPLC.

Results

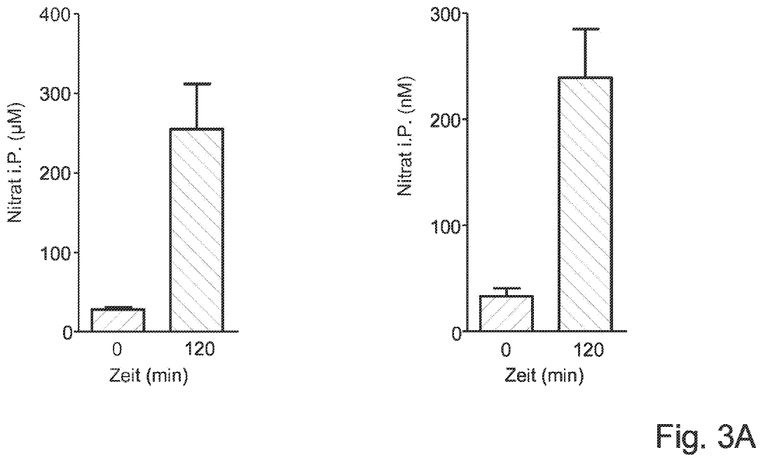

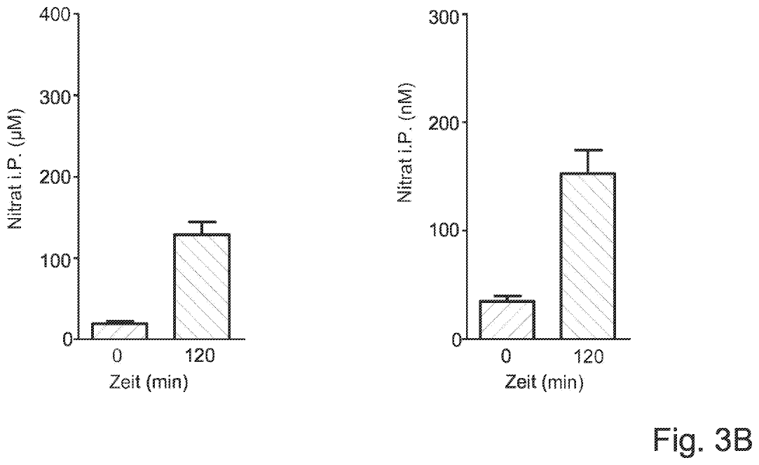

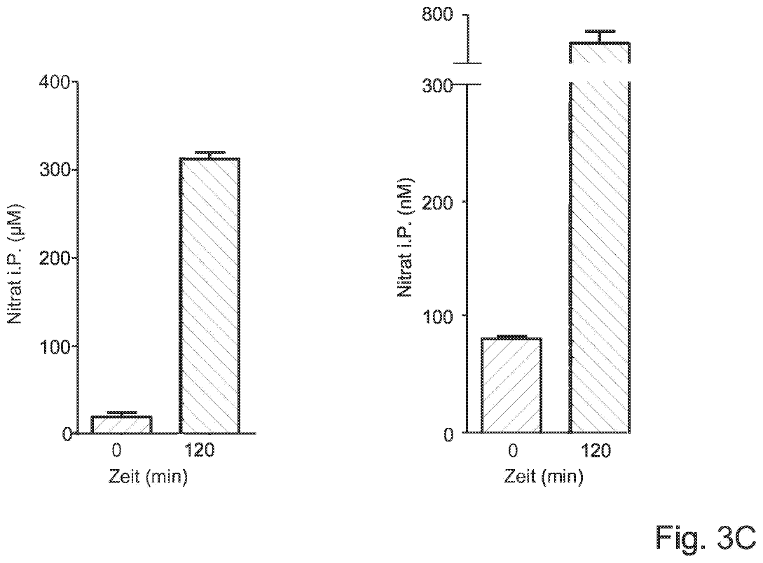

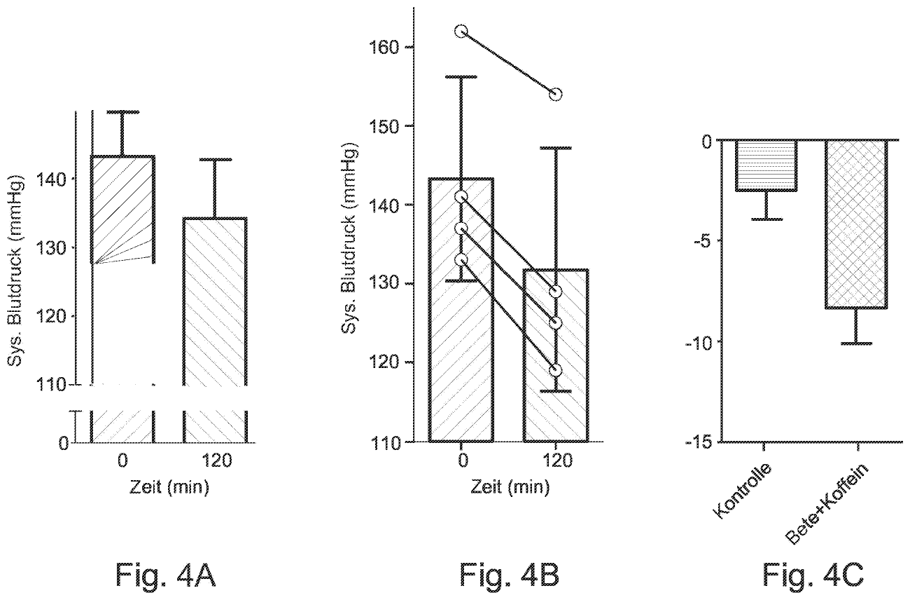

The results of the blood test with regard to the average nitrate and nitrite concentration in the plasma of the test subjects are shown in FIG. 3A (group 3, composition C, beet), FIG. 3B (group 1, composition A, white beet) and FIG. 3C (group 2, composition B, yellow beet). FIG. 4 shows the specific effects of composition A (group 1, white beet) on systolic blood pressure. FIG. 4A shows the averaged blood pressure values obtained during blood pressure measurements before consumption of the composition and two hours after consumption. FIG. 4B shows the results of a further evaluation, which captures the relationship between basal blood pressure (initial blood pressure) and achieved minimum blood pressure values. Finally, FIG. 4C shows the average or mean blood pressure reduction in subjects who received composition A compared to a (negative) control group.

Composition C with (red) beet juice led to a significant increase in both nitrate and nitrite concentrations in the plasma of the test subjects two hours after consumption, as shown in FIG. 3A. Composition A with white beet juice as the nitrate source also led to an increase in blood nitrate and nitrite levels (see FIG. 3B). Finally, a significant increase in the nitrate and nitrite concentration in the plasma of the test subjects can also be achieved with composition B based on yellow beet juice (cf. FIG. 3C). The compositions according to the invention thus provide a suitable basis for actually increasing the concentration of nitric oxide, which acts as a vasodilator in the blood and has a vasodilating and thus blood pressure-lowering effect.

To further investigate the antihypertensive effect of the compositions according to the invention, composition A based on white beet juice was used:

FIG. 4A shows the systolic blood pressure values obtained during the blood pressure measurements before and two hours after consumption of composition A and averaged over all subjects in the group. Before consumption of the compositions, the subjects had an average systolic blood pressure of approximately 143 mmHg, i.e. values in the arterial hypertension range. Two hours after consumption of composition A, the values had fallen to an average of around 132 mmHg, i.e. blood pressure was reduced to values in the high normal range.

In FIG. 4B, the basal blood pressure values of individual subjects and the minimum values measured in the same subjects are correlated. This correlation shows, on the one hand, that a reduction in blood pressure could be achieved in each individual subject on the basis of the composition according to the invention. On the other hand, it is evident that the minimum blood pressure measured in each case depends on the basal blood pressure. Overall, the systolic blood pressure can thus be reliably lowered by about 10 to 15 mmHg by the compositions according to the invention. An excessive drop in blood pressure, which can be accompanied by undesirable symptoms such as listlessness, dizziness, etc., does not occur.

FIG. 4C also compares the results of the average blood pressure reduction (difference in syst. blood pressure [in mmHg]) in a control group that had not received any blood pressure-lowering composition and the subjects who had received composition A. In the control group, only an average blood pressure reduction of 2 mmHg was measured, whereas composition A had led to an average blood pressure reduction of approximately 8 mmHg. In the control group, an average blood pressure reduction of only 2.5 mmHg was measured, whereas composition A had led to an average blood pressure reduction of approximately 8 mmHg.

4. Summary

Based on the above, it can be seen that in the context of the present invention, an antihypertensive composition is provided which is based on well-tolerated, natural ingredients, namely plant nitrate sources, antioxidants and minerals, and which leads to a significant reduction in systolic blood pressure. Taking into account that the composition is based on natural ingredients, it is essentially free from undesirable side effects that may occur in connection with drug therapy for hypertension.

Particularly surprisingly, the combined use of a stimulating or concentration-enhancing substance based on caffeine also does not lead to a reversal of the antihypertensive effect.

As a result, a side-effect-free composition is provided which is suitable for non-medicinal blood pressure lowering and also exhibits further physiological properties, in particular a concentration-promoting and stimulating effect, which is further supplemented by antioxidant effects and minerals vital to the body.

Another advantage of the present invention or the compositions according to the invention is that the blood pressure-lowering effect occurs only when the systolic blood pressure is above the normal range, i.e. too high, or in the upper normal range, i.e. between 120 and 130 mmHg. At low blood pressure, on the other hand, there is no further reduction in blood pressure, i.e., there is no risk of hypotension. Thus, the composition according to the invention is also suitable for persons with low or normal blood pressure, so that they can benefit from the further advantageous properties of the composition according to the invention, in particular the concentration-promoting and stimulating effect as well as the antioxidant effects and the supplementation with minerals.

1-15. (canceled)

16. A composition, comprising:
(a) at least one nitrate source; and
(b) caffeine;
wherein the composition promotes concentration, has a stimulating effect, and/or lowers arterial blood pressure.

17. The composition according to claim 16, wherein the at least one nitrate source contains nitrate in an amount of 1-10,000 mg, based on the total weight of NO₃ and/or NO₃⁻per serving or application unit of the composition.

18. The composition according to claim 16, wherein the at least one nitrate source is selected from vegetable nitrate sources, salts of nitric acid, esters of nitric acid and combinations thereof.

19. The composition according to claim 18, wherein the at least one nitrate source is a vegetable nitrate source selected from plants of the group of Spinacia oleracea (vegetable spinach), Raphanus (radish), Beta vulgaris (common turnip), Brassica rapa (turnip), Diplotaxis tenuifolia (arugula), Lepidium (cress) and Valerianella (lamb's lettuce).

20. The composition according to claim 18, wherein the at least one nitrate source is a vegetable nitrate source derived from the yellow, red and/or white cultivated form of Beta vulgaris (common turnip).

21. The composition according to claim 18, wherein the at least one nitrate source is a vegetable nitrate source in the form of liquid plant components or solid plant components.

22. The composition according to claim 17, wherein the at least one nitrate source is a salt of nitric acid selected from alkali metal and alkaline earth metal salts of nitric acid.

23. The composition according to claim 16, wherein the amount of caffeine in the composition is in an amount of 1-1,500 mg per portion or application unit of the composition.

24. The composition according to claim 16, wherein a ratio of the at least one nitrate source and the caffeine is from 1:10-50:1 based on the total weight of the composition.

25. The composition according to claim 16, further comprising at least one mineral selected from calcium, chlorine, potassium, magnesium, sodium, phosphorus, sulfur, iron, iodine, copper, manganese, molybdenum, selenium, zinc and combinations thereof.

26. The composition according to claim 16, further comprising at least one vitamin or provitamin selected from vitamin A, vitamin B, vitamin C, vitamin D, vitamin E, vitamin K and combinations thereof.

27. The composition according to claim 16, further comprising sulforaphane.

28. The composition according to claim 27, wherein the amount of sulforaphane in the composition is 0.01-500 mg.

29. The composition according to claim 16, further comprising chewing gum base as an excipient.

30. The composition according to claim 16, further comprising at least one solid carrier or matrix material.

31. The composition according to claim 16, further comprising at least one liquid carrier material.

32. The composition according to claim 16, which is formulated for oral delivery via consumption or topical application.

33. The composition according to claim 16, wherein the composition is prepared as a beverage, bar, pastry, hard caramel, chewing gum, toothpaste or mouthwash.

34. A method of treating arterial hypertension, increasing energy level or increasing concentration, comprising:
administering a composition comprising:
(a) at least one nitrate source; and
(b) caffeine;
to a subject in need thereof.

35. The method of claim 34, wherein the composition is formulated as a foodstuff, food supplement, or hygiene article.
